(19) United States
(12) Patent Application Publication
(10) Pub. No.: US 2007/0264672 A1
(43) Pub. Date: Nov. 15, 2007

(54) DEVELOPMENT OF A NOVEL ASSAY FOR MGMT (METHYL GUANINE METHYL TRANSFERASE)

(76) Inventors: Bimalendu Dasmahapatra, Nutley, NJ (US); Mark Demma, Edison, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/745,033

(22) Filed: May 7, 2007

Related U.S. Application Data
(60) Provisional application No. 60/798,914, filed on May 9, 2006.

(51) Int. Cl. G01N 33/53; C12M 3/00

(52) U.S. Cl. 435/7.5; 435/287.2

(57) ABSTRACT

The present invention provides improved methods for assessing the level of MGMT activity in a variety of biological preparations. MGMT, a DNA repair enzyme, can reduce the chemotherapeutic efficacy of alkylating agents by repairing the damage that alkylating agents do to tumor cell DNA. The methods of the present invention can be used, inter alia, to measure MGMT levels and to thereby predict the clinical response to alkylating agents. The present invention includes three preferred assays for assessment of MGMT activity: (1) the immunoassay technique, (2) the labeled O⁶—BG technique, and (3) the fluorescence polarization technique. Kits useful for the performance of such assays are also provided.

### REFERENCE TO RELATED APPLICATION

This application claims the benefit of priority to U.S. Provisional Patent Application 60/798,914 filed May 9, 2006, the entire disclosure of the priority application is hereby incorporated by reference.

### FIELD OF THE INVENTION

The present invention relates to the DNA repair protein known as O⁶-methylguanine-DNA methyltransferase (“MGMT”) and, in particular, to improved assays for assessing MGMT activity in a variety of biological preparations. These assays can be used to predict the clinical response to chemotherapeutic treatment with alkylating agents for the treatment of certain tumor types.

### BACKGROUND OF THE INVENTION

Discussion or citation of a reference herein shall not be construed as an admission that such reference is prior art to the present invention.

Chemotherapeutic efficacy, the ability of chemotherapy to eradicate tumor cells without causing lethal host toxicity, depends on drug selectivity. One class of anticancer drugs, alkylating agents, binds to DNA and adds alkyl groups at various positions of bases, including the O⁶ position of guanine, thereby structurally distorting the DNA helical structure. This prevents DNA transcription and translation, resulting in cell death. In this way, alkylating agents inhibit cellular proliferation.

In normal cells, the damaging action of alkylating agents can be repaired by cellular DNA repair enzymes, in particular O⁶-methylguanine-DNA methyltransferase (“MGMT”), also known as O⁶-alkylguanine-DNA-alkyltransferase (“AGAT”). MGMT repairs O⁶-alkylguanine by transferring the alkyl group to MGMT's active center. This simultaneously restores the DNA and inactivates MGMT. The methylated form of MGMT then becomes detached from the DNA and is targeted for degradation by ubiquitination. Srivenugopal et al., Biochemistry 35:1328-1334 (1996). Thus, MGMT is a suicide enzyme, capable of acting only once.

The level of MGMT varies in normal and tumor cells, even among tumors of the same type. How well a cell can repair the effects of an alkylating agent depends on the level of MGMT in that cell and its rate of resynthesis. MGMT is, therefore, a crucial biomarker of tumor resistance to alkylating agents. Nagel et al., Anal. Biochem. 321:38-43 (2003). Measurement of MGMT activity in biopsy specimens would allow prediction of a patient's clinical resistance to alkylating agents. Aida et al., Carcinogenesis 8:1219-1223 (1987); Fujio et at, Carcinogenesis 10.351-356 (1989).

Identification of a tumor with elevated MGMT levels would suggest intervention with agents such as O⁶ benzylguanine (“O⁶—BG”), Patrin-2, streptozotocin and other benzyloxypyrimidines, which can deplete MGMT levels and restore sensitivity to alkylating agents. Friedman et al., Journal of Clinical Oncology 16:3851-3857 (1998). For example, in the human carcinoma HT-29 cell line, exposure to O⁶—BG or streptozotocin inhibited MGMT activity for 48 hours, which resulted in potentiation of BCNU-induced cytotoxicity by three logs over that observed with BCNU alone. Depletion of MGMT with O⁶—BG has also enhanced chemotherapy-induced cytotoxicity in glioma cell lines. Jaeckle et al., Journal of Clinical Oncology 16:3310-3315 (1998).

Current methods for measuring the enzymatic activity of MGMT protein in cell or tissue extracts include techniques described by: Myrnes et al., Carcinogenesis 5:1061-1064 (1984); Futscher et al., Cancer Comm. 1:65-73 (11989); Kreklau et al., J. Pharmacol. Exper. Ther. 297(2):524-530 (2001); and Nagel et al., Anal. Biochem. 321(1):3843 (2003), the entire disclosures of which are incorporated herein in their entireties.

The first of these methods involves measuring the transfer of a [³H]-methyl group from substrate DNA to MGMT protein. Essentially, substrate DNA containing a [³H]-methyl group in the O⁶-position of guanine is incubated with a cell or tissue extract under protein-limiting conditions until the transfer reaction is complete. Excess substrate DNA is hydrolyzed to acid solubility and separated from methylated radioactive protein by filtration or centrifugation. Radioactivity in the residual protein is measured by liquid scintillation counting. See, e.g., Myrnes et al., Carcinogenesis 5:1061-1064 (1984).

Another method involves the use of substrate DNA that has been radioactively end-labeled and contains O⁶-methylguanine, typically in a restriction enzyme site. Transfer of the O⁶-methyl group to MGMT allows the restriction enzyme to cleave the DNA substrate, producing a radiolabeled fragment. The amount of the radiolabeled fragment produced is proportional to the level of MGMT activity. See, e.g., Futscher et al., Cancer Comm. 1:65-73 (1989); Kreklau et al., J. Pharmacol. Exp. Ther. 291:1269-1275 (1999).

A variation of this technique calls for fluorimetric end-labeling of the DNA substrate instead of radiolabeling. This modification results in a fluorescently labeled digestion cleavage product that may be detected and quantitated using a fluorescence imaging system. Kreklau et al., J. Pharmacol. Exp. Ther. 297(2):524-530 (2001).

A fourth method is based on the reaction of MGMT with an O⁶—BG derivative incorporated into an oligonucleotide. The O⁶—BG derivative is characterized by a biotin group linked to the 4-position of the benzyl group. When MGMT reacts with this DNA substrate, MGMT is biotinylated and may be detected in an ELISA. Nagel at al., Anal. Biochem. 321(1):3843 (2003).

Each of these four techniques requires the preparation of labeled DNA oligonucleotides for use as a substrate for MGMT. The process for preparing this substrate DNA is both time-consuming and inefficient. Thus, there is a need in the art for an assay for MGMT activity that does not require the use of a DNA substrate.

In addition, the most commonly used techniques for measuring MGMT activity rely on radiolabels to tag the DNA substrate. The use of such radioisotopes is costly and associated with environmental hazards. Simple, rapid assays for MGMT activity that could be carried out without radioisotopes, therefore, would be advantageous.

In view of the deleterious side effects of most chemotherapeutic drugs, and the ineffectiveness of certain drugs for various treatments, there is a great need for treatments targeted to specific classes of patients. Improved methods for predicting whether a patient is likely to respond well to treatment with alkylating agents would be a welcome contribution to the art. The present invention provides just such methods.

### SUMMARY OF THE INVENTION

The present invention addresses, inter alia, the need in the art for rapid and convenient assays for assessing the level of MGMT activity in a variety of biological preparations. Such assays may be used to predict a patient's clinical response to chemotherapeutic treatment with alkylating agents. Such information is useful, for example, to tailor chemotherapeutic treatment to a patient's specific needs.

The rapid assays of the present invention are technically advantageous in comparison to previous assays for MGMT activity. First, because the assays of this invention do not require the preparation of a DNA substrate, they are technically simpler and significantly more efficient than prior art assays. Second, they may be carried out without the use of radioisotopes, increasing safety and reducing the environmental hazards and costs associated with waste disposal. Moreover, the MGMT activity assays of the present invention can provide both qualitative and quantitative detection of MGMT activity.

The assays of this invention that use fluorescently labeled O⁶—BG are additionally advantageous in that they require less cell lysate than prior art assays.

The fluorescence polarization assays of the present invention offer several additional advantages. This is a homogeneous technique (e.g., a “single addition” or “mix and read” assay) that does not require any manipulation after the reaction is initiated, for example, the separation of reactants from products. This saves time and reduces the potential for artifacts. The fluorescence polarization technique also allows real-time measurements to be made directly in solution, and the assay signal can be monitored continuously, providing real-time kinetic data on MGMT enzymatic activity. Because instrumentation is available that can measure fluorescence polarization in high-density microplates very rapidly and with great precision, it is well-suited for high-throughput screening.

Because they allow for more effective, safer implementation of alkylating agents in cancer patients, the rapid MGMT activity assays of the present invention will have a significant social and economic impact.

The present invention provides a method for assessing the level of MGMT activity in a biological sample comprising: (a) contacting the sample with O⁶—BG such that any MGMT in the sample can react with O⁶—BG to form a complex; (b) detecting and quantitating the complex; and (c) based on the quantity of the detected complex, determining the level of MGMT activity in the sample.

In some embodiments, detecting step (b) comprises immunoassay detection.

In some embodiments, the immunoassay detection comprises the steps of: (1) coating the wells of a plate with the complex; (2) treating the plate with an anti-MGMT antibody such that the anti-MGMT antibody binds to the complex; and (3) detecting the bound anti-MGMT antibody in a fluorescent, enzymatic, chemiluminescent or radioactive assay system. In more specific embodiments, the O⁶—BG is labeled with biotin and the plate is coated with avidin.

In some embodiments, the immunoassay detection comprises the steps of: (1) coating the wells of a plate with the complex; (2) treating the plate with an anti-MGMT antibody such that the anti-MGMT antibody binds to the complex; (3) treating the plate with a secondary antibody such that the secondary antibody binds to the anti-MGMT antibody; and (4) detecting the bound secondary antibody in a fluorescent, enzymatic, chemiluminescent or radioactive assay system. In more specific embodiments, the O⁶—BG is labeled with biotin and the plate is coated with avidin.

The present invention also provides a method for assessing the level of MGMT activity in a biological sample comprising: (a) contacting the sample with O⁶—BG such that any MGMT in the sample can react with O⁶—BG to form a complex, wherein the benzyl group of O⁶—BG is labeled; (b) separating unreacted O⁶—BG from the complex; (c) detecting and quantitating the complex; and (d) based on the quantity of the detected complex, determining the level of MGMT activity in the sample.

In some embodiments, the separation of unreacted O⁶—BG in step (b) comprises precipitation of the complex with acetone.

In some embodiments, the separation of unreacted O⁶—BG in step (b) comprises: (1) adding the sample mixture to a plate coated with an anti-MGMT antibody, and (2) washing the plate to remove unreacted O⁶—BG.

In some embodiments, the O⁶—BG label is fluorescent, enzymatic, chemiluminescent or radioactive.

In more specific embodiments, the O⁶—BG label is fluorescent and is selected from the group consisting of rare earth chelates, fluorescein, rhodamine, dansyl, dansyl chloride, umbelliferone, Lissamine, phycoerythrin, phycocyanin, allophycocyanin, o-phthaldehyde, fluorescamine, Texas Red, BODIPY, Alexa Fluors, Dyomics Dyes, Quasar Dyes, CY-dyes, and derivatives thereof.

In more specific embodiments, the O⁶—BG label is enzymatic and is selected from the group consisting of luciferases; 2,3-dihydrophthalazinediones; malate dehydrogenase; urease; peroxidases; alkaline phosphatase, beta-galactosidase; glucoamylase; lysozyme; saccharide oxidases; heterocyclic oxidases; acetylcholinesterase; lactoperoxidase and microperoxidase.

In more specific embodiments, the O⁶—BG label is chemiluminescent and is selected from the group consisting of luminol, isoluminol, theromatic acridinium ester, imidazole, acridinium salt, oxalate ester, luciferin and aequorin.

In more specific embodiments, the O⁶—BG label is radioactive and is selected from the group consisting of ³⁵S, ¹⁴C, ³H, ³²P, ¹²⁵I, ¹³¹I, ¹⁵N, ⁹⁰Y, ⁹⁹Tc and ¹¹¹In. The present invention also provides a method for assessing the level of MGMT activity in a biological sample comprising: (a) contacting the sample with a known quantity of fluorescently labeled O⁶—BG such that any MGMT in the sample can react with O⁶—BG to form a complex; (b) detecting fluorescence polarization indicative of complex formation, wherein the fluorescence polarization measurement indicates the quantity of the complex; and (c) based on the quantity of the complex, determining the level of MGMT activity in the sample.

In more specific embodiments, the fluorescent label is selected from the group consisting of rare earth chelates, fluorescein, rhodamine, dansyl, dansyl chloride, umbelliferone, Lissamine, phycoerythrin, phycocyanin, allophycocyanin, o-phthaldehyde, fluorescamine, Texas Red, BODIPY, Alexa Fluors, Dyomics Dyes, Quasar Dyes, CY-dyes, and derivatives thereof.

The present invention also provides a method for predicting a chemotherapeutic efficacy of an alkylating agent in a patient in need thereof comprising assessing the level of MGMT activity in a biological sample from the patient by: (a) contacting the sample with O⁶—BG such that any MGMT in the sample can react with O⁶—BG to form a complex; (b) detecting and quantitating the complex; and (c) based on the quantity of the detected complex, determining the level of MGMT activity in the sample; wherein the predicted chemotherapeutic efficacy is inversely related to the level of MGMT activity in the sample.

In some embodiments, detecting step (b) comprises immunoassay detection.

In some embodiments, the immunoassay detection comprises the steps of: (1) coating the wells of a plate with the complex; (2) treating the plate with an anti-MGMT antibody such that the anti-MGMT antibody binds to the complex; and (3) detecting the bound anti-MGMT antibody in a fluorescent, enzymatic, chemiluminescent or radioactive assay system. In more specific embodiments, the O⁶—BG is labeled with biotin and the plate is coated with avidin.

In some embodiments, the immunoassay detection comprises the steps of: (1) coating the wells of a plate with the complex; (2) treating the plate with an anti-MGMT antibody such that the anti-MGMT antibody binds to the complex; (3) treating the plate with a secondary antibody such that the secondary antibody binds to the anti-MGMT antibody; and (4) detecting the bound secondary antibody in a fluorescent, enzymatic, chemiluminescent or radioactive assay system. In more specific embodiments, the O⁶—BG is labeled with biotin and the plate is coated with avidin.

The present invention also provides a method for predicting a chemotherapeutic efficacy of an alkylating agent in a patient in need thereof comprising assessing the level of MGMT activity in a biological sample from the patient by: (a) contacting the sample with O⁶—BG such that any MGMT in the sample can react with O⁶—BG to form a complex, wherein the benzyl group of O⁶—BG is labeled; (b) separating unreacted O⁶—BG from the complex; (c) detecting and quantitating the complex; and (d) based on the quantity of the detected complex, determining the level of MGMT activity in the sample; wherein the predicted chemotherapeutic efficacy is inversely related to the level of MGMT activity in the sample.

In some embodiments, the separation of unreacted O⁶—BG in step (b) comprises precipitation of the complex with acetone.

In some embodiments, the separation of unreacted O⁶—BG in step (b) comprises: (1) adding the sample mixture to a plate coated with an anti-MGMT antibody, and (2) washing the plate to remove unreacted O⁶—BG.

In some embodiments, the O⁶—BG label is fluorescent, enzymatic, chemiluminescent or radioactive.

In more specific embodiments, the O⁶—BG label is fluorescent and is selected from the group consisting of rare earth chelates, fluorescein, rhodamine, dansyl, dansyl chloride, umbelliferone, Lissamine, phycoerythrin, phycocyanin, allophycocyanin, o-phthaldehyde, fluorescamine, Texas Red, BODIPY, Alexa Fluors, Dyomics Dyes, Quasar Dyes, CY-dyes, and derivatives thereof.

In more specific embodiments, the O⁶—BG label is enzymatic and is selected from the group consisting of luciferases; 2,3-dihydrophthalazinediones; malate dehydrogenase; urease; peroxidases; alkaline phosphatase; beta-galactosidase; glucoamylase; lysozyme; saccharide oxidases; heterocyclic oxidases; acetylcholinesterase; lactoperoxidase and microperoxidase.

In more specific embodiments, the O⁶—BG label is chemiluminescent and is selected from the group consisting of luminol, isoluminol, theromatic acridinium ester, imidazole, acridinium salt, oxalate ester, luciferin and aequorin.

In more specific embodiments, the O⁶—BG label is radioactive and is selected from the group consisting of ³⁵S, ¹⁴C, ³H, ³²P, ¹²⁵I, ¹³¹I, ¹⁵N, ⁹⁰Y, ⁹⁹Tc and ¹¹¹In.

The present invention also provides a method for predicting a chemotherapeutic efficacy of an alkylating agent in a patient in need thereof comprising assessing the level of MGMT activity in a biological sample from the patient by: (a) contacting the sample with a known quantity of fluorescently labeled O⁶—BG such that any MGMT in the sample can react with O⁶—BG to form a complex; (b) detecting fluorescence polarization indicative of complex formation, wherein the fluorescence polarization measurement indicates the quantity of the complex; and (c) based on the quantity of the complex, determining the level of MGMT activity in the sample, wherein the predicted chemotherapeutic efficacy is inversely related to the level of MGMT activity in the sample.

In more specific embodiments, the fluorescent label is selected from the group consisting of rare earth chelates, fluorescein, rhodamine, dansyl, dansyl chloride, umbelliferone, Lissamine, phycoerythrin, phycocyanin, allophycocyanin, o-phthaldehyde, fluorescamine, Texas Red, BODIPY, Alexa Fluors, Dyomics Dyes, Quasar Dyes, CY-dyes, and derivatives thereof.

The present invention also provides any of the methods described above, comprising the additional step of comparing the level of MGMT activity in the sample to that in (a) a control sample exhibiting a high level of MGMT activity, (b) a control sample exhibiting a low level of MGMT activity, or (c) both (a) and (b).

In some embodiments, the control sample exhibiting a high level of MGMT activity is selected from the group consisting of an HT29 sample, a Capan-1 sample or a Capan-2 sample. In some embodiments, the control sample exhibiting a low level of MGMT activity is an SNB19 sample.

The present invention also provides any of the methods described above, wherein the biological sample is a tissue or cell sample from lung, breast, ovary, stomach, pancreas, larynx, esophagus, testes, liver, parotid, biliary tract, colon, rectum, cervix, uterus, endometrium, kidney, spleen, bladder, prostate, thyroid, lymph node, pituitary, eye, brain, oral cavity, skin, bone, bone marrow, semen, stool, or a fraction or component thereof.

The present invention also provides any of the methods described above, wherein the biological sample is a tumor biopsy sample and the tumor type is prostate, breast, lung, pancreatic, colorectal, urinary system, NHL, melanoma, cervical, leukemia, oral cavity, ovarian, testicular, esophageal, liver, kidney, spleen, head and neck, carcinoma, sarcoma, lymphoma, mycosis fungoides or malignant glioma.

In some embodiments, the tumor biopsy sample is from bone marrow or lymph node and is from a patient suffering from leukemia. In some embodiments, the tumor biopsy sample is from a high-grade tumor.

The present invention also provides any of the methods described above, wherein the alkylating agent is temozolomide, dacarbazine, busulfan, thiotepa, hydroxymethylmelamine, hexamethylmelamine, cyclophosphamide, mechlorethamine, uramustine, melphalan, chlorambucil, carmustine, streptozocin, lomustine, semustine, ifosfamide, porfiromycin, procarbazine, mitocycin C, cisplatin or carboplatin. In a preferred embodiment, the alkylating agent is temozolomide.

The present invention also provides a kit for conducting one or more methods selected from any of the methods described above. In some embodiments, the kit comprises: (1) reagents used in the methods of the invention; and (2) instructions for carrying out the methods of the invention.

### BRIEF DESCRIPTION OF THE FIGURES

FIG. 1A is a schematic of the mechanism by which O⁶ BG inhibits MGMT. O⁶—BG reacts with MGMT by covalent transfer of O⁶—BG's benzyl group to MGMT's active site cysteine. This reaction yields MGMT with S-benzylcysteine at its active site and stoichiometric amounts of guanine.

FIG. 1B depicts the steps of an MGMT assay with labeled O⁶—BG. In this assay, the amount of MGMT in a cell sample is detected by contacting cell lysate derived from the sample with O⁶—BG that has been labeled with fluorescein, using acetone to precipitate MGMT-bound O⁶—BG, and measuring the quantity of MGMT-bound O⁶—BG with a fluorometer.

FIG. 1C depicts the steps of an MGMT immunoassay. In this assay, the amount of MGMT in a cell sample is detected by contacting cell lysate derived from the sample with biotinylated O⁶—BG, performing an ELISA using an anti-MGMT antibody and a secondary antibody, and measuring the quantity of MGMT-bound O⁶—BG with a plate reader.

FIG. 2 provides the results of experiments in which various concentrations of O⁶—BG labeled with fluorescein (“FI—O⁶—BG”) were read in a fluorescent plate reader. The resulting standard curve for FI—O⁶—BG indicates that FI—O⁶—BG was accurately detected. The slope of the curve is 0.1369, and the Y-intercept is 90.24 when X=0.0.

FIG. 3 depicts the experimental results of an MGMT assay with fluorescently labeled O⁶—BG. The experiment was used to determine the optimal amount of cell lysate to use in this assay. Various amounts of HT29 cell lysate were incubated with 20 μM FI—O⁶—BG overnight at 4° C. Each mixture was then precipitated with 2.5 vol. cold acetone, incubated on ice for 1 hour, and centrifuged at 15,000 rpm for 15 minutes. Each pellet was dried, resuspended in buffer and read in a fluorescent reader. MGMT activity was determined to be 1.25 nmoles/mg protein.

FIG. 4 depicts the experimental results of an MGMT assay with fluorescently labeled O⁶—BG. The experiment was used to determine the optimal amount of FI—O⁶—BG to use in this type of assay. 10 μg of HT29 cell lysate was incubated with various concentrations of FI—O⁶—BG overnight at 4° C. The mixture was then precipitated with 2.5 vol. cold acetone, incubated on ice for 1 hour, and centrifuged at 15,000 rpm for 15 minutes. The pellet was dried, resuspended in buffer and read in a fluorescent reader. The results indicate that the saturation point was reached using approximately 50,000 fmoles FI—O⁶—BG.

FIG. 5A provides the results of MGMT assays with fluorescently labeled O⁶—BG and DAOY, SNB19, SNB75, WiDr, H1299, KLE, Capan 2 and Capan 1 cell lysates. 10 μg of each cell lysate was incubated with 20 μM FI—O⁶—BG overnight at 4° C. The mixture was then precipitated with 2.5 vol. cold acetone, incubated on ice for 1 hour, and centrifuged at 15,000 rpm for 15 minutes. The pellet was dried, resuspended in buffer and read in a fluorescent reader. The assay results clearly show different levels of MGMT activity in the different cell lines. For example, Capan 2 and Capan 1 cells exhibited the highest levels of MGMT activity, while SNB75 and SNB19 cells exhibited the lowest.

FIG. 5B provides the results of a Western blot performed on DAOY, SNB19, SNB75, WiDr, H1299, KLE, Capan 2 and Capan 1 cells using an anti-MGMT antibody. The results are consistent with the results of the MGMT assays with fluorescently labeled O⁶—BG described in FIG. 5A.

FIG. 6 depicts the experimental results of an MGMT ELISA. Different amounts of recombinant MGMT protein were incubated with 500 nM of biotinylated O⁶—BG overnight, then detected using an anti-MGMT antibody and a goat anti-mouse secondary antibody labeled with horseradish peroxidase. The resulting standard curve for MGMT indicates that the MGMT ELISA accurately detects MGMT. 100,000 luminescence counts equals 42.5 pmoles of MGMT. The slope of the curve is 1958+/−40.17. The Y-intercept when X=0.0 is 16750+/−3811.

FIG. 7 shows experimental results of MGMT ELISAs run using various amounts of biotinylated O⁶—BG and four different quantities of MGMT protein (0 μg, 5 μg, 10 μg and 20 μg). The experiments were performed to determine the optimum amount of biotinylated O⁶—BG to use in an MGMT ELISA.

FIG. 8A provides experimental results of MGMT ELISAs run on various amounts of Capan 1 cell lysate, DAOY cell lysate and SNB19 cell lysate. In each assay, cell lysate was exposed to 500 nM biotinylated O⁶—BG. MGMT-bound O⁶—BG was captured using avidin-coated plates, then detected using an anti-MGMT antibody and a goat anti-mouse secondary antibody conjugated to horseradish peroxidase. Capan 1 cells exhibited the most MGMT activity, followed by DAOY cells. SNB19 cells exhibited minimal MGMT activity.

FIG. 8B provides the results of a Western blot performed on DAOY, SNB19 and Capan 1 cells using an anti-MGMT antibody. The results are consistent with the results of the MGMT ELISAs described in FIG. 8A.

FIG. 9 depicts MGMT activity in 50 μg of cell lysate as measured by prior art radioactive assays on DAOY, Widr, SNB75, SNB19, Capan 1 and Capan 2 cells. The results are consistent with the results of the MGMT ELISAs described in FIG. 8A.

### DETAILED DESCRIPTION OF THE INVENTION

Described herein are functional assays useful for assessing MGMT activity in a variety of biological preparations. These assays can be used to predict a patient's clinical response to treatment with alkylating agents. Such information is useful, for example, to tailor chemotherapeutic treatment to a patient's specific needs.

### Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, Cold Spring Harbor Laboratory Press, Cold Spring Harbor, N.Y. (3d Edition 2001); Ausubel et al., Current Protocols in Molecular Biology, John Wiley & Sons, Inc. (2006); and Harlow and Lane, Antibodies: A Laboratory Manual, Cold Spring Harbor Laboratory Press, Cold Spring Harbor, N.Y. (1988), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As used herein, the following definitions shall apply unless otherwise indicated:

Throughout this specification and claims, the word “comprise,” or variations such as “comprises” or “comprising,” will be understood to imply the inclusion of a stated integer or group of integers but not the exclusion of any other integer or group of integers.

The term “chemotherapeutic efficacy” is intended to mean mitigating or alleviating a cell proliferative disorder in a mammal such as a human.

The term “alkylating agent” refers to a class of anticancer drugs that bind to DNA and add alkyl groups at various positions of bases, including the O⁶ position of guanine, thereby structurally distorting the DNA helical structure. This prevents DNA transcription and translation, resulting in cell death and inhibiting cellular proliferation. Examples of alkylating agents include but are not limited to temozolomide, dacarbazine, busulfan, thiotepa, hydroxymethylmelamine, hexamethylmelamine, cyclophosphamide, mechlorethamine, uramustine, melphalan, chlorambucil, carmustine, streptozocin, lomustine, semustine, ifosfamide, porfiromycin, procarbazine, mitocycin C, cisplatin and carboplatin.

Temozolomide is an alkylating agent available from Schering Corp. under the trade name of Temodar®. Temodar® Capsules for oral administration contain temozolomide, an imidazotetrazine derivative. The chemical name of temozolomide is 3,4-dihydro-3-methyl-4-oxoimidazo[5,1-d]-as-tetrazine-8-carboxamide. Its metabolite is known as MTIC. Alkylation (methylation) occurs mainly at the O⁶, N⁷ and N³ positions of guanine.

Temodar® (temozolomide) Capsules are currently indicated in the United States for the treatment of adult patients with newly diagnosed gliobastoma multiforme as well as refractory anaplastic astrocytoma, i.e., patients at first relapse who have experienced disease progression on a drug regimen containing a nitrosourea and procarbazine. Temodar® is currently approved in Europe for the treatment of patients with malignant glioma, such as glioblastoma multiforme or anaplastic astrocytoma showing recurrence or progression after standard therapy.

The term “patient” includes human and veterinary subjects.

The term “biological sample” refers to a fraction of cells or tissue, or a biological fluid such as whole blood. The sample can be obtained as or isolated from, for example, lung, breast, ovary, stomach, pancreas, larynx, esophagus, testes, liver, parotid, biliary tract, colon, rectum, cervix, uterus, endometrium, kidney, spleen, bladder, prostate, thyroid, lymph node, pituitary, eye, brain, oral cavity, skin, bone, bone marrow, semen, stool, a fraction or component of any of the above, or any other biological specimen containing MGMT protein. The sample to be assayed can come from biopsies, but can also be obtained from tissue culture or other laboratory preparations. The sample can be pre-treated and can be prepared in any convenient medium that does not interfere with the assay. An aqueous medium is preferred.

Where the biological sample is a tumor biopsy sample, the tumor type may be, for example, prostate, breast, lung, pancreatic, colorectal, urinary system, NHL, melanoma, cervical, leukemia, oral cavity, ovarian, testicular, esophageal, liver, kidney, spleen, head and neck, carcinoma, sarcoma, lymphoma, mycosis fungoides or malignant glioma. Carcinomas include, for example, squamous cell carcinoma, adenocarcinoma and small cell carcinoma. The tumor may be a high-grade tumor.

The term “O⁶-methylguanine-DNA methyltransferase” and the abbreviation “MGMT” are each intended to mean the family of homologous proteins, specific forms of which are found in most living organisms, which have the ability to transfer alkyl groups, for example, methyl groups, from the O⁶ position of guanine in alkylated DNA to a cysteine residue of their own polypeptide chain. Depending on the context, this term and abbreviation are also used to refer to individual members of the family.

cDNA for human MGMT has been cloned and the DNA and amino acid sequences published in Tano et al., PNAS 87:686-690 (1990), the relevant portions of which are incorporated herein by reference. See also Hayakawa et al., J. Mo. Biol. 213:739-747 (1991); Rydberg et al., J. Boil. Chem. 265:9563-9569 (1990); and Von Wronski et al., J. Biol. Chem. 266:1064-1070 (1991), the relevant portions of which are incorporated herein by reference.

The term “O⁶—BG” refers to O⁶ benzylguanine.

The term “MGMT-bound O⁶—BG or “MGMT-O⁶—BG complex” refers to MGMT with S-benzylcysteine at its active site. This complex is the product of the reaction of O⁶—BG with MGMT.

The term “ELISA” refers to an enzyme-linked immunosorbent assay that employs an antigen or antibody bound to a solid phase and an enzyme-antibody or enzyme-antigen conjugate to detect and quantify the amount of an antigen or antibody present in a sample. An enzyme-antibody conjugate or an enzyme-antigen conjugate is then used to detect the bound complex. The conjugated enzyme cleaves a substrate to generate a colored reaction product that can be detected spectrophotometrically. The absorbance of the colored solution is proportional to the amount of the colored reaction product. A review of ELISA is found in Ausubel et al., Current Protocols in Molecular Biology, John Wiley & Sons, Inc. (2006), the disclosure of which is hereby incorporated by reference.

The term “antibody” refers both to monoclonal antibodies, which are a substantially homogeneous population and to polyclonal antibodies, which are heterogeneous populations. Polyclonal antibodies are derived from the sera of animals immunized with an antigen, Monoclonal antibodies (MAbs) to specific antigens may be obtained by methods known to those skilled in the art. See, for example, U.S. Pat. No. 4,376,110. Such antibodies may be of any immunoglobulin class including IgG, IgM, IgE, IgA, IgD and any subclass thereof. Methods of making and detecting detectably labeled antibodies or their functional derivatives are well known to those of ordinary skill in the art.

The term “antibody” is also meant to include both intact molecules as well as fragments thereof, such as, for example, Fab and F(ab′)2, which are capable of binding antigen. Fab and F(ab′)2 fragments lack the Fc fragment of intact antibody. It will be appreciated that Fab and F(ab′)2 and other fragments of the antibodies useful in the present invention may be used for the detection and quantitation of MGMT according to the methods disclosed herein in the same manner as an intact antibody. Such fragments are typically produced by proteolytic cleavage, using enzymes such as papain (to produce Fab fragments) or pepsin (to produce F(ab′)2 fragments).

An antibody is said to be “capable of binding” a molecule if it is capable of specifically reacting with the molecule to thereby bind the molecule to the antibody. The term “epitope” is meant to refer to that portion of any molecule capable of being bound by an antibody that can also be recognized by that antibody. Epitopic determinants usually consist of chemically active surface groupings of molecules such as amino acids or sugar side chains and have specific three-dimensional structural characteristics as well as specific charge characteristics.

An “antigen” is a molecule capable of being bound by an antibody that is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies that may be evoked by other antigens.

The antibodies, or fragments of antibodies, useful in the present invention may be used to quantitatively or qualitatively detect the presence of MGMT in a biological sample.

The term “label” as used herein refers to a detectable compound or composition which is conjugated directly or indirectly with a molecule, such as O⁶—BG, an anti-MGMT antibody or a secondary antibody. Various labels may be employed. In some embodiments, the label may be detectable by itself (e.g., fluorescent labels or radioisotope labels). In other embodiments, the label may be an enzymatic label that catalyzes a chemical alteration of a substrate compound or composition that is detectable. In preferred embodiments, the label is a fluorescent label or an enzymatic label that catalyzes a color change of a non-radioactive color reagent.

Generally, O⁶—BG and/or an antibody will be labeled either directly or indirectly with a detectable label. In some embodiments, labels are attached by spacer arms of various lengths to reduce potential steric hindrance. Numerous labels are available, which can be generally grouped into the following categories:

(a) Fluorescent labels, such as rare earth chelates (europium chelates), fluorescein, rhodamine, dansyl, dansyl chloride, umbelliferone, Lissamine, phycoerythrin, phycocyanin, allophycocyanin, o-phthaldehyde, fluorescamine, Texas Red, BODIPY, Alexa Fluors, Dyomics Dyes, Quasar Dyes, CY-dyes, and derivatives thereof. The fluorescent labels can be conjugated to O⁶—BG or to an antibody using the techniques disclosed in Current Protocols in Immunology, John Wiley & Sons, Inc. (2006), for example. Fluorescence can be quantified using a fluorometer (e.g., Nynatech).

(b) Enzymatic labels, such as luciferases (e.g., firefly luciferase and bacterial luciferase; U.S. Pat. No. 4,737,456), 2,3-dihydrophthalazinediones, malate dehydrogenase, urease, peroxidase such as horseradish peroxidase (HRP), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), acetylcholinesterase, lactoperoxidase, microperoxidase, and the like. U.S. Pat. No. 4,275,149 provides a review of some of the enzyme-substrate labels that are available. The enzyme, when later exposed to its substrate, will generally catalyze a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a Dynatech ML3000 chemiluminometer, for example) or donates energy to a fluorescent acceptor. Techniques for conjugating enzymes to antibodies are described in O'Sullivan and Marks, Methods Enzymol 73:147-166 (1981) and Current Protocols in Immunology, John Wiley & Sons, Inc. (2006).

Examples of enzyme-substrate combinations include, for example:

(i) Horseradish peroxidase (HRPO) with hydrogen peroxidase as a substrate, wherein the hydrogen peroxidase oxidizes a dye precursor (e.g., orthophenylene diamine [OPDD or 3,3′5,5′-tetramethyl benzidine hydrochloride [TMB]).

(ii) alkaline phosphatase (AP) with para-Nitrophenyl phosphate as chromogenic substrate.

(iii) beta-D-galactosidase (beta-D-Gal) with a chromogenic substrate (e.g., p-nitrophenyl-.beta.-D-galactosidase) or fluorogenic substrate 4-methylumbelliferyl-.beta.-D-galactosidase.

Numerous other enzyme-substrate combinations are available to those skilled in the art. For a general review of these, see U.S. Pat. Nos. 4,275,149 and 4,318,980.

(c) Chemiluminescent labels, such as luminol, isoluminol, theromatic acridinium ester, imidazole, acridinium salt and oxalate ester, and bioluminescent labels, such as luciferin and aequorin. The presence of a chemiluminescent label is determined by detecting the luminescence that arises during the course of a chemical reaction. Bioluminescence is a type of chemiluminescence found in biological systems in which a catalytic protein increases the efficiency of the chemiluminescent reaction.

(d) Radioisotopes, such as ³⁵S, ¹⁴C, ³H, ³²P, ¹²⁵I, ¹³¹I, ¹⁵N, ⁹⁰Y, ⁹⁹Tc and ¹¹¹In. O⁶—BG or an antibody can be labeled with the radioisotope using the techniques described in Current Protocols in Immunology, John Wiley & Sons, Inc. (2006), for example. The radiolabel can be detected and measured by using any of the currently available counting procedures, for example, by using a scintillation counter, a gamma counter or by autoradiography.

Sometimes, the label is indirectly conjugated with the antibody. The skilled artisan will be aware of various techniques for achieving this. For example, O⁶—BG or an antibody can be conjugated with biotin and any of the broad categories of labels mentioned above can be conjugated with avidin, or vice versa. Biotin binds selectively to avidin and, thus, the label can be conjugated with O⁶—BG or an antibody in this indirect manner. See, e.g., Current Protocols in Immunology, John Wiley & Sons, Inc. (2006), for a review of techniques involving biotin-avidin conjugation. Alternatively, to achieve indirect conjugation of the label with O⁶—BG or an antibody, the antibody is conjugated with a small hapten (e.g. digoxin) and one of the different types of labels mentioned above is conjugated with an anti-hapten antibody (e.g., anti-digoxin antibody). Thus, indirect conjugation of the label with the antibody can be achieved.

In a preferred embodiment of the invention, the anti-MGMT antibody need not be labeled, and the presence thereof can be detected using a labeled secondary antibody (e.g., anti-mouse anti-MGMT antibody conjugated with HRP).

Various labeling techniques are described in Morrison, Methods Enzymol. 32b:103-109 (1974); Syvanen et al., J. Biol. Chem. 248:3762-3768 (1973); and Bolton and Hunter, Biochem. J. 133:529-539 (1973), hereby incorporated by reference.

### Assays

The present invention includes assay methods by which the level of active MGMT in a biological sample may be determined precisely, rapidly and conveniently. Assays for MGMT activity in tumor tissue are especially important because the results of the assays can be used to select appropriate treatment protocols. As greater understanding of the function and distribution of MGMT in normal and diseased cells is obtained through the use of the assays of the invention, further predictive applications of the invention will be developed. For example, the quantitative assays of the present invention can help physicians evaluate whether treatment of a patient with an alkylating agent is likely to benefit the patient.

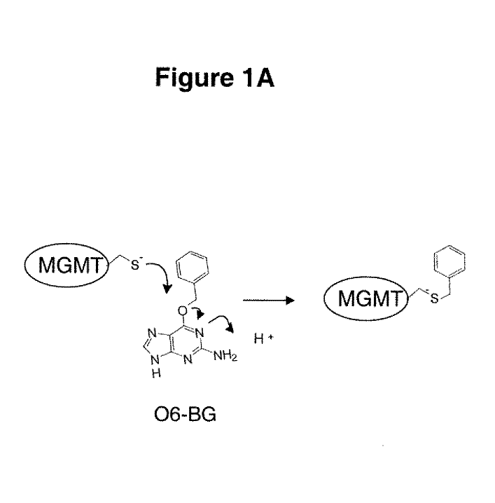

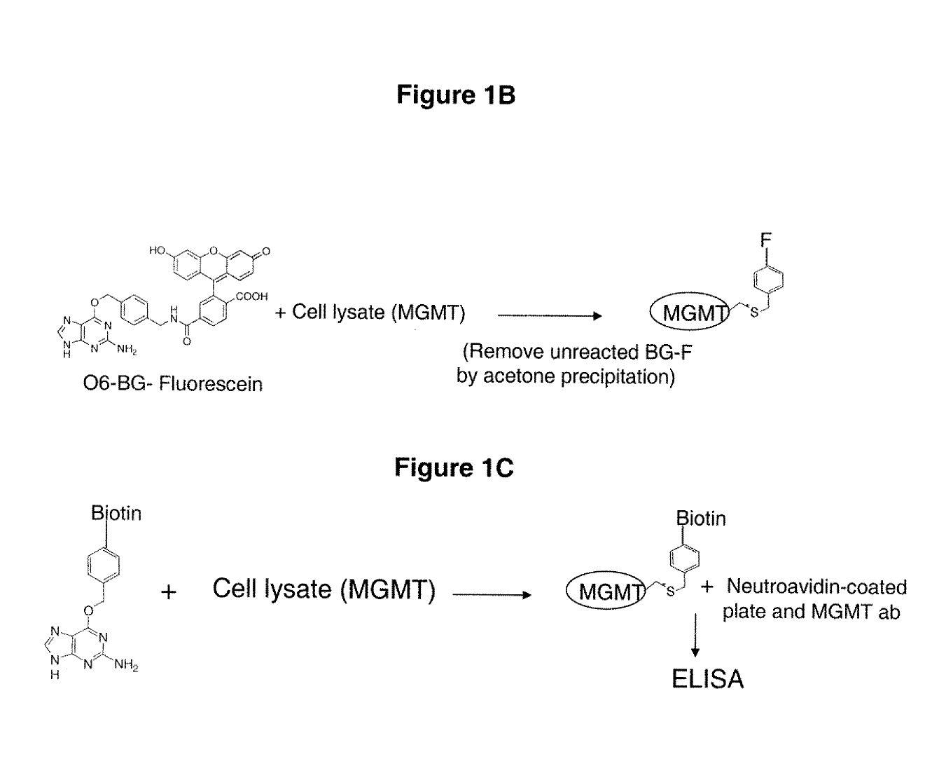

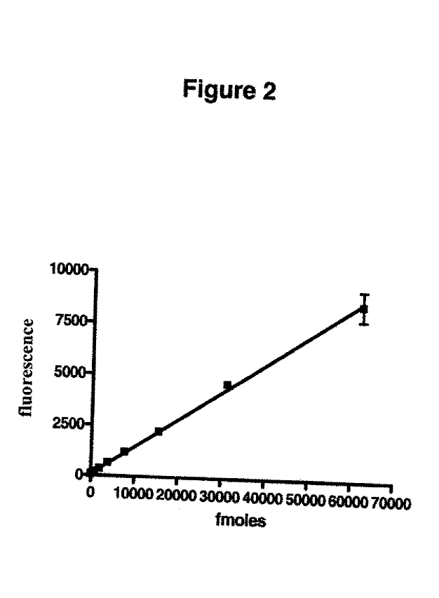

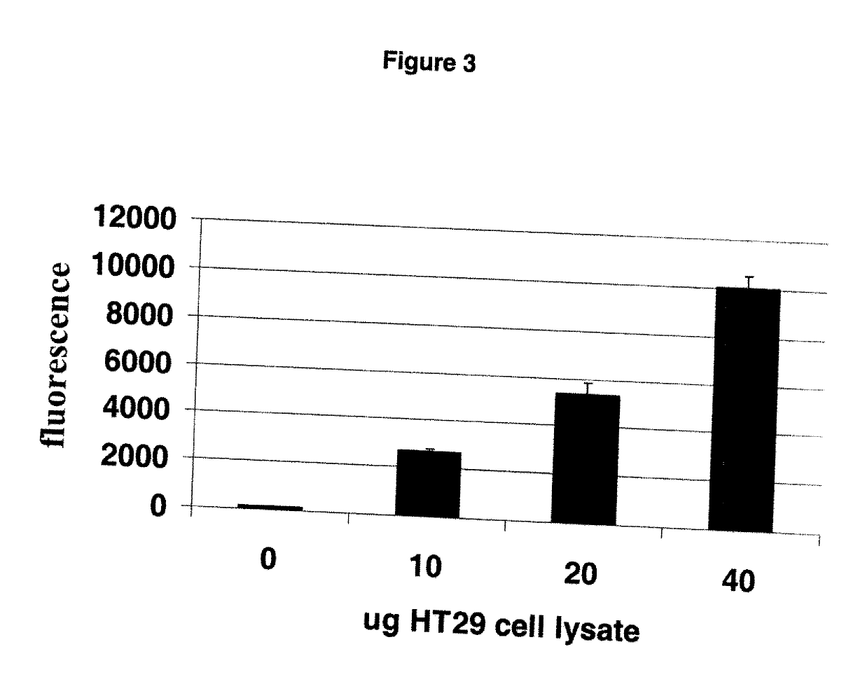

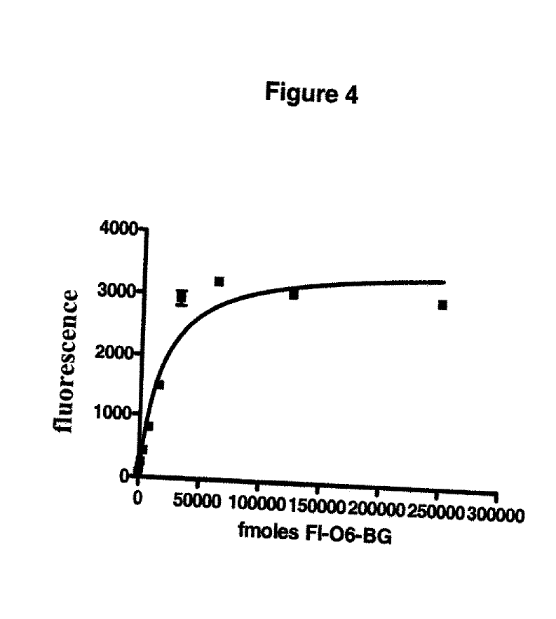

Essentially, the processes of this invention comprise incubating or otherwise exposing the sample to be tested to O⁶—BG and then detecting the presence of a reaction product. O⁶—BG will react with any MGMT in the test sample by covalent transfer of O⁶—BG's benzyl group to MGMT's active site cysteine. This reaction yields MGMT with S-benzylcysteine at its active site (referred to herein as “MGMT-bound O⁶—BG”, the “MGMT-O⁶—BG complex”, or “the complex”) and stoichiometric amounts of guanine. Pegg et al., Biochemistry 32:11998-12006 (1993); Gerson, Nature Reviews: Cancer 4.296-307 (2004). A schematic diagram of this reaction is provided in FIG. 1A.

Thus, the present invention provides various methods for assessing the level of MGMT activity in a biological sample comprising:

(a) contacting the sample with O⁶—BG such that any MGMT in the sample can react with O⁶—BG to form a complex;

(b) detecting and quantitating the complex; and

(c) based on the quantity of the detected complex, determining the level of MGMT activity in the sample.

Those skilled in the art will recognize that there are many variations of this basic procedure. These include the use of, for example, immunofluorescence, ELISA, RIA, fluorescence polarization, etc.

Suitable labels for O⁶—BG, anti-MGMT antibodies or secondary antibodies have been disclosed supre and include various fluorescent materials, enzymatic materials, chemiluminescent materials and radioactive materials.

The present invention includes three preferred rapid assays for assessment of MGMT activity: (1) the immunoassay technique, (2) the labeled O⁶—BG technique, and (3) the fluorescence polarization technique.

Each of the assays of the present invention can be used to assess the level of MGMT activity in a biological sample. This information can be used to predict the chemotherapeutic efficacy of an alkylating agent in a patient in need thereof. Generally, the predicted chemotherapeutic efficacy is inversely related to the level of MGMT activity in a sample. For example, where the level of MGMT activity in a sample is low, the patient from whom the sample derives would be expected to benefit from treatment with an alkylating agent. Where the level of MGMT activity in a sample is elevated, the patient who provided the sample would be unlikely to respond well to treatment with an alkylating agent. Such cases would suggest pretreatment with O⁶—BG, Patrin-2, streptozotocin, other benzyloxypyrimidines, or other agents that can deplete MGMT levels and restore sensitivity to alkylating agents prior to treatment with an alkylating agent.

Immunoassay Technique

The present invention provides an immunoassay technique for detecting and quantitating reaction of MGMT with O⁶—BG.

In some embodiments, the immunoassay technique for assessing the level of MGMT activity in a biological sample generally comprises the following steps:
(a) contacting the sample with O⁶—BG such that any MGMT in the sample can react with O⁶—BG to form a complex;
(b) detecting and quantitating the complex by:
   (1) coating the wells of a plate with the complex;
   (2) treating the plate with an anti-MGMT antibody such that the anti-MGMT antibody binds to the complex; and
   (3) detecting the bound anti-MGMT antibody in a fluorescent, enzymatic, chemiluminescent or radioactive assay system; and
(c) based on the quantity of the detected complex, determining the level of MGMT activity in the sample.

In these embodiments, the anti-MGMT antibody is conjugated to the fluorescent, enzymatic, chemiluminescent or radioactive label. The label can be bound to the anti-MGMT antibody directly, or a conjugating molecule can be conjugated to the anti-MGMT antibody, and the label can subsequently be bound to the anti-MGMT antibody via the conjugating molecule. After excess anti-MGMT antibody has been washed away, the complex can be detected by detecting the label.

In some embodiments, the immunoassay technique for assessing the level of MGMT activity in a biological sample generally comprises the following steps:
(a) contacting the sample with O⁶—BG such that any MGMT in the sample can react with O⁶—BG to form a complex;
(b) detecting and quantitating the complex by:
   (1) coating the wells of a plate with the complex;
   (2) treating the plate with an anti-MGMT antibody such that the anti-MGMT antibody binds to the complex;
   (3) treating the plate with a secondary antibody such that the secondary antibody binds to the anti-MGMT antibody; and
   (4) detecting the bound secondary antibody in a fluorescent, enzymatic, chemiluminescent or radioactive assay system; and
(c) based on the quantity of the detected complex, determining the level of MGMT activity in the sample.

In these embodiments, a secondary antibody binds to the anti-MGMT antibody, and it is the secondary antibody that is conjugated to the fluorescent, enzymatic, chemiluminescent or radioactive label. The label can be bound to the secondary antibody directly, or a conjugating molecule can be conjugated to the secondary antibody, and the label can subsequently be bound to the secondary antibody via the conjugating molecule. After excess secondary antibody has been washed away, the complex can be detected by detecting the label.

In some embodiments of the immunoassay technique, the plate may be coated with the complex by first coating the plate with a capture agent that will bind specifically to the complex. In a preferred embodiment, the wells of the plate are coated with avidin, e.g., streptavidin or neutroavidin, and O⁶—BG is labeled with biotin.

In a preferred embodiment, the immunoassay is an “ELISA”, which refers to an enzyme-linked immunosorbent assay. Such an ELISA involves capturing the MGMT-O⁶—BG complex to a solid phase (usually the well of an ELISA microtiter plate). An enzyme conjugated to an anti-MGMT antibody or a secondary antibody is then used to detect the bound complex. The conjugated enzyme catalyzes a color change of a non-radioactive color reagent. Accordingly, the complex can be detected by a subsequent color change of the reagent. The absorbance of the colored solution in individual microtiter wells is proportional to the amount of the MGMT-O⁶—BG complex. Thus, an MGMT ELISA can both detect and quantify the amount of MGMT present in a sample. Suitable enzymatic labels for anti-MGMT antibodies and secondary antibodies have been disclosed supra.

In some embodiments, the MGMT ELISA technique for assessing the level of MGMT activity in a biological sample generally comprises the following steps:

(a) The sample is contacted with O⁶—BG such that any MGMT in the sample can react with O⁶—BG to form a complex.

(b) A solid phase (usually a well of an ELISA microtiter plate) is coated with a capture agent (often avidin) which binds specifically to the MGMT-O⁶—BG complex (where, for example, O⁶—BG is labeled with biotin).

(c) The biological sample is then exposed to, or contacted with, the adhering capture agent so that the complex adheres to (or is captured by) the solid phase.

(d) A washing step is then carried out to remove unbound portions of the biological sample, leaving the captured complex.

(e) The adhering or captured complex is then exposed to, or contacted with, an anti-MGMT antibody. This allows the anti-MGMT antibody to bind to the captured complex.

(f) A washing step is then carried out to remove unbound anti-MGMT antibody.

(g) The adhering or captured complex is then exposed to, or contacted with, a secondary antibody that binds to the anti-MGMT antibody. In the preferred embodiment, the secondary antibody is conjugated (directly or indirectly) to an enzyme that catalyses a color change of a non-radioactive color reagent. Accordingly, the complex can be detected by a subsequent color change of the reagent. The enzyme can be bound to the secondary antibody directly, or a conjugating molecule can be conjugated to the secondary antibody, and the enzyme can be subsequently bound to the secondary antibody via the conjugating molecule.

(h) A washing step is then carried out to remove unbound secondary antibody.

(i) Finally, binding of the secondary antibody to the captured complex is measured, for example, by a color change in the color reagent.

In some embodiments, the anti-MGMT antibody is itself conjugated to the enzyme that catalyses a color change of a non-radioactive color reagent. In this case, use of a secondary antibody is not required. After excess anti-MGMT antibody has been washed away, the complex can be detected by a subsequent color change of the reagent.

Labeled O⁶—BG Tech

The present invention also provides a labeled O⁶—BG technique for detecting and quantitating reaction of MGMT with O⁶—BG.

In some embodiments, the labeled O⁶—BG technique for assessing the level of MGMT activity in a biological sample generally comprises the following steps:
(a) contacting the sample with O⁶—BG such that any MGMT in the sample can react with O⁶—BG to form a complex, wherein the benzyl group of O⁶—BG is labeled;
(b) separating unreacted O⁶—BG from the complex;
(c) detecting and quantitating the complex; and
(d) based on the quantity of the detected complex, determining the level of MGMT activity in the sample.

In some embodiments, separation of unreacted O⁶—BG from the complex in step (b) comprises precipitation of the complex with acetone.

In some embodiments, separation of unreacted O⁶—BG from the complex in step (b) comprises:
(1) adding the sample mixture to a plate coated with an anti-MGMT antibody, and
(2) washing the plate to remove unreacted O⁶—BG.

In some embodiments, the O⁶—BG label used in the labeled O⁶—BG technique is fluorescent, enzymatic, chemiluminescent or radioactive.

In a preferred embodiment, the O⁶—BG label is fluorescent and is selected from the group consisting of rare earth chelates, fluorescein, rhodamine, dansyl, dansyl chloride, umbelliferone, Lissamine, phycoerythrin, phycocyanin, allophycocyanin, o-phthaldehyde, fluorescamine, Texas Red, BODIPY, Alexa Fluors, Dyomics Dyes, Quasar Dyes, CY-dyes, and derivatives thereof.

In some embodiments, the O⁶—BG label is enzymatic and is selected from the group consisting of luciferases; 2,3-dihydrophthalazinediones; malate dehydrogenase; urease; peroxidases; alkaline phosphatase; beta-galactosidase; glucoamylase; lysozyme; saccharide oxidases; heterocyclic oxidases; acetylcholinesterase; lactoperoxidase and microperoxidase.

In some embodiments, the O⁶—BG label is chemiluminescent and is selected from the group consisting of luminol, isoluminol, theromatic acridinium ester, imidazole, acridinium salt, oxalate ester, luciferin and aequorin.

In some embodiments, the O⁶—BG label is radioactive and is selected from the group consisting of ³⁵S, ¹⁴C, ³H, ³²P, ¹²⁵I, ¹³¹I, ¹⁵N, ⁹⁰Y, ⁹⁹Tc and ¹¹¹In.

Fluorescence Polarization Technique

The present invention additionally provides a fluorescence polarization technique for detecting and quantitating reaction of MGMT with O⁶—BG. Specifically, reaction of MGMT with fluorescently labeled O⁶—BG to form a complex can provide a detectable fluorescence polarization signal that indicates the presence and level of the complex in the sample.

Fluorescence polarization is used to study molecular interactions by monitoring changes in the apparent size of fluorescently labeled or inherently fluorescent molecules. It provides a direct, nearly instantaneous measure of the fluorescent molecule's bound/free ratio.

When a polarized light excites the fluorescent substance in a sample, the polarization of the fluorescent light emitted depends on the excited dipoles in the sample. For samples with structure orientation preference, if the excitation polarization is aligned with the preferred structure orientation, then the fluorescence emission light will have a dominant polarization orientation with relatively strong intensity. If the excitation polarization is perpendicular to the preferred structure orientation, then the intensity of the fluorescence emission light will be relatively weak. For samples with no structure orientation preference, the fluorescence emission light will have randomized polarization direction, i.e., it will be non-polarized.

The degree of fluorescence polarization of a fluorescent molecule is a reflection of its molecular weight. Fluorescence polarization is therefore a useful detection method for homogeneous assays in which the starting reagents and products differ significantly in molecular weight. Hsu et al., Biotechniques 31(3):560, 562, 564-568, passim. (2001).

Complexes, such as those formed by MGMT associating with O⁶—BG's fluorescently labeled benzyl group, have higher polarization values than uncomplexed, fluorescently labeled O⁶—BG. Accordingly, the fluorescence emission light will be highly polarized for O⁶—BG that has reacted with MGMT relative to the signal generated by unreacted O⁶—BG. In this way, fluorescence polarization techniques can be used to detect and measure complex formation between MGMT and fluorescently labeled O⁶—BG.

Any of a variety of known dyes can be used in fluorescence polarization methods, including fluorescein dyes, cyanine dyes, dansyl dyes, and polyazaindacene dyes, such as 4,4-difluoro-4-bora-3a,4a-diaza-s-indacene (BODIPY) dyes (Molecular Probes, Eugene, Oreg., see, e.g., U.S. Pat. Nos. 6,323,186 and 6,005,113). Fluorescence polarization assays are known in the art and selection of dyes and assay conditions can be determined according to the assay design. A review of fluorescence polarization techniques is found in the Fluorescence Polarization Technical Resource Guide (3d Edition, PanVera/Invitrogen Corp. 2004), the disclosure of which is hereby incorporated by reference.

In some embodiments, the fluorescence polarization technique for assessing the level of MGMT activity in a biological sample generally comprises the following steps:
(a) contacting the sample with a known quantity of fluorescently labeled O⁶—BG such that any MGMT in the sample can react with O⁶—BG to form a complex;
(b) detecting fluorescence polarization indicative of complex formation, wherein the fluorescence polarization measurement indicates the quantity of the complex; and
(c) based on the quantity of the complex, determining the level of MGMT activity in the sample.

In some embodiments, the fluorescent label is selected from the group consisting of rare earth chelates, fluorescein, rhodamine, dansyl, dansyl chloride, umbelliferone, Lissamine, phycoerythrin, phycocyanin, allophycocyanin, o-phthaldehyde, fluorescamine, Texas Red, BODIPY, Alexa Fluors, Dyomics Dyes, Quasar Dyes, CY-dyes, and derivatives thereof.

Each of the assays of the present invention may comprise the additional step of comparing the level of MGMT activity in the sample to that in

(a) a control sample exhibiting a high level of MGMT activity,

(b) a control sample exhibiting a low level of MGMT activity, or

(c) both (a) and (b).

In some embodiments of the comparison step, the control sample exhibiting a high level of MGMT activity is selected from the group consisting of an HT29 sample, a Capan-1 sample or a Capan-2 sample.

In some embodiments of the comparison step, the control sample exhibiting a low level of MGMT activity is an SNB19 sample.

As more is learned about the distribution of MGMT in normal and diseased cells, additional biological samples useful as control samples will be identified or developed. It is appreciated that such control samples will also be useful in the methods and kits of the present invention.

Each of the assays of the present invention is also ideally suited for the preparation of a kit. Thus, the present invention also provides a kit comprising reagents and instructions for conducting one or more of the assays of the present invention. Such a kit may comprise a carrier means being compartmentalized to receive in close confinement therewith one or more container means such as plates, vials, tubes and the like, each of said container means comprising the separate elements of the assay A kit may include diagnostic or therapeutic agents, as well as instructions for use of the kit in a diagnostic or therapeutic method.

### EXAMPLES

In order that this invention may be better understood, the following examples are set forth. These examples are for purposes of illustration only and are not to be construed as limiting the scope of the invention in any manner. Any composition disclosed in the example along with any disclosed method is part of the present invention.

### Example 1

### The immunoassay Technique

In this study, we detected the amount of MGMT in a cell sample by contacting cell lysate derived from the sample with biotinylated O⁶—BG, performing an ELISA using an anti-MGMT antibody and a secondary antibody, and measuring the quantity of MGMT-bound O⁶—BG with a plate reader. For a schematic diagram of the assay procedure, see FIG. 1C.

Materials and Methods

Exponentially growing cells are treated with trypsin and washed with PBS. Pelleted cells are resuspended with buffer (40 mM Tris-HCl, pH=8.0, 10% glycerol, 1 mM EDTA, 1 mM OTT, and protease inhibitors (Roche protease inhibitor cocktail tablets)) and lysed by sonication. Cell lysate is then centrifuged at 10,000K for 10 min.

Pierce Reacti-bind neutroavidin-coated plates are washed with Tris buffered saline with 0.5% Triton X-100 three times, and then blocked for a half hour with Pierce superblock. 90 μl cell lysate is then added to the cell, together with 10 μl of 5 μM biotinylated O⁶—BG (Covalys) (final concentration, 500 nM), and incubated overnight at 4° C. After the incubation, the plates are washed three times with TBST to remove unbound cell lysate components, and the plate is treated with an anti-MGMT antibody (mouse anti-human MGMT, Pharmagin (Becton-Dickenson) catalog number 557045) at a 1:500 dilution. The antibody incubation occurs at room temperature for one hour with shaking. The plate is washed with TBST three times to remove unbound antibody, and then treated with a secondary antibody (goat anti-mouse immunoglobulin coupled to horseradish peroxidase, Jackson Immunoresearch catalog number 115-035-062) at a 1:2000 dilution. The plate is then incubated with shaking for one hour at room temperature. The plate is washed three times with TBST to remove unbound secondary antibody, developed with either ABST or chemiluminescent substrate, and read with the appropriate plate reader.

Results

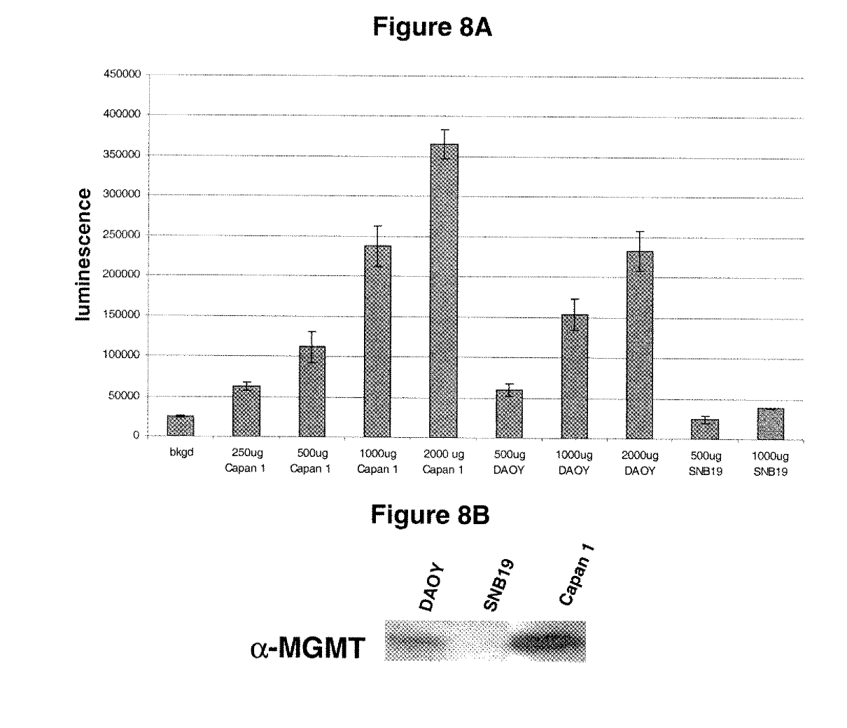

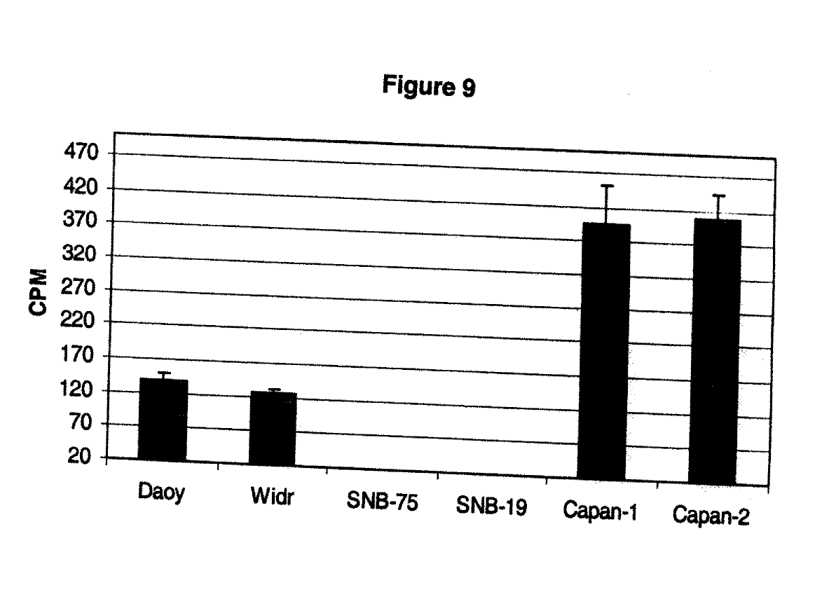

Results of this assay using various amounts of Capan 1 cell lysate, DAOY cell lysate and SNB19 cell lysate are provided in FIG. 8A. The assay results clearly show different levels of MGMT activity in the different cell lines, with Capan 1 cells exhibiting the highest levels of MGMT activity and SNB19 cells exhibiting the least. The assay results are consistent with a Western blot performed on these cells using an anti-MGMT antibody (see FIG. 8B). The assay results are also consistent with results obtained using prior art radioactive assays (see FIG. 9).

### Example 2

### The Labeled O⁶—BG Technique

In this study, we detected the amount of MGMT in a cell sample by contacting cell lysate derived from the sample with fluorescently labeled O⁶—BG, using acetone to precipitate MGMT-bound O⁶—BG, and measuring the quantity of MGMT-bound O⁶—BG with a fluorometer. For a schematic diagram of the assay procedure, see FIG. 1B.

Materials and Methods

Exponentially growing cells are treated with trypsin and washed with PBS. Pelleted cells are resuspended with buffer (40 mM Tris-HCl, pH=8.0, 10% glycerol, 1 mM EDTA, 1 mM DTT, and protease inhibitors (Roche protease inhibitor cocktail tablets)) and lysed by sonication. Cell lysate is then centrifuged at 10,000K for 10 min.

Fluorescent O⁶—BG (Covalys) at 20 μM is incubated with 10 μg cell lysate at 4° C. overnight in 40 mM Tris, pH=8.0, 1 mM EDTA, and 1 mM DTT. After overnight incubation, the mixture is precipitated with 2.5 volumes ice-cold acetone, and incubated on ice for one hour. The mixture is then centrifuged in a microfuge at 15,000 rpm for 15 min. The supernatant is aspirated off, and the pellet is dried using a speed-vac. The precipitate is then resuspended in 100 μl of Tris buffer and read using a fluorometer with an excitation filter at 485 nm and an emission filter at 535 nm.

Results

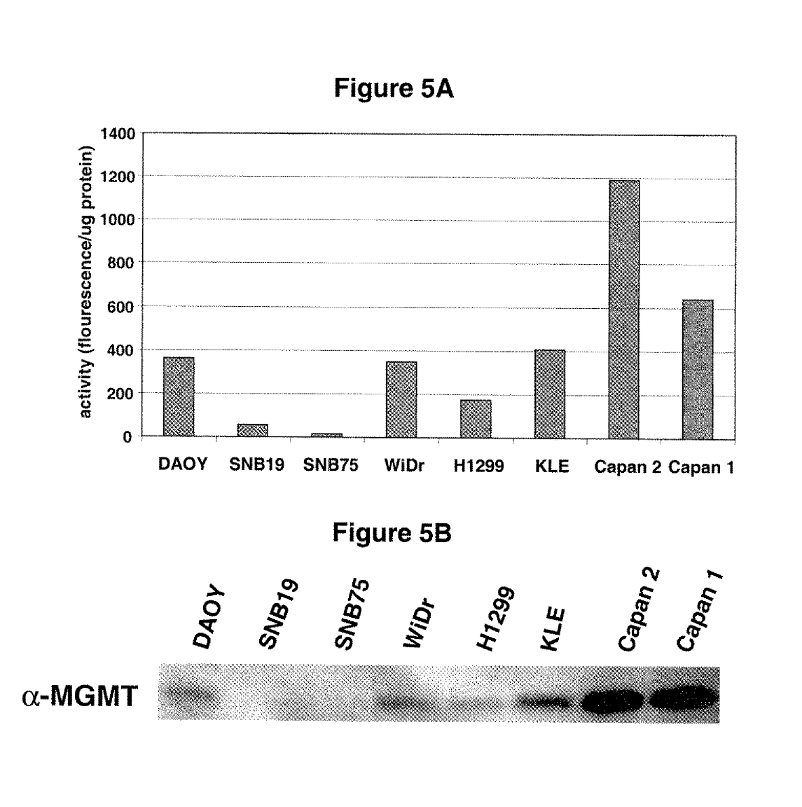

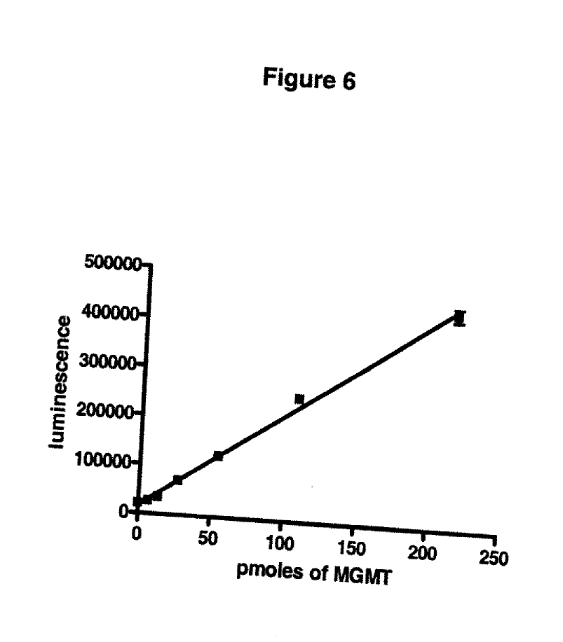

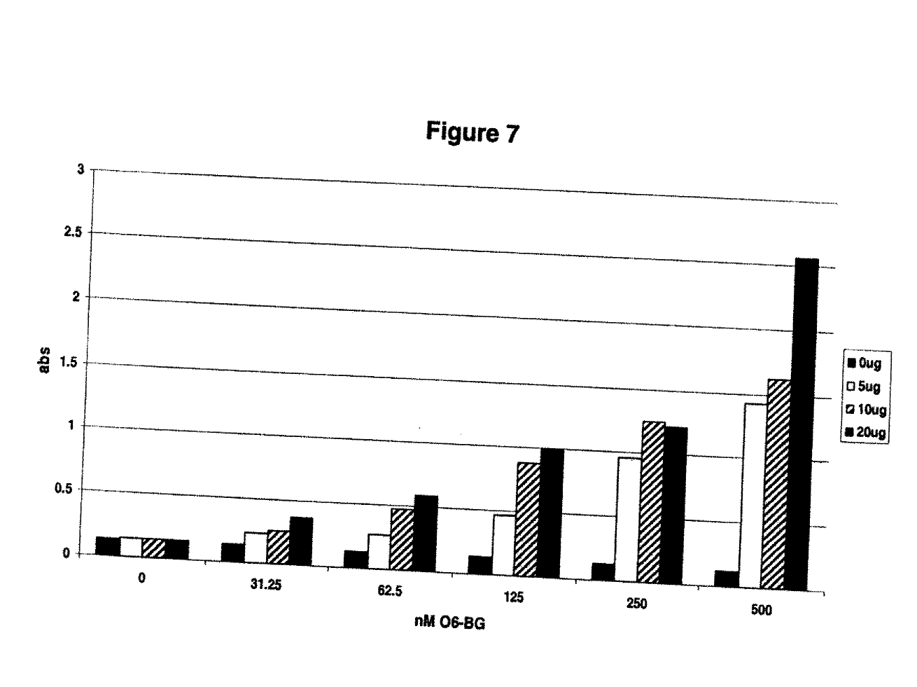

Results of this assay using 10 μg each of DAOY, SNB19, SNB75, WiDr, H1299, KLE, Capan 2 and Capan 1 cell lysates are provided in FIG. 5A. The assay results clearly show different levels of MGMT activity in the different cell lines. For example, Capan 2 and Capan 1 cells exhibited the highest levels of MGMT activity, while SNB75 and SNB19 cells exhibited the lowest. The assay results are consistent with a Western blot performed on these cells using an anti-MGMT antibody (see FIG. 5B).

### Example 3

### The Fluorescence Polarization Technique

One can detect the amount of MGMT in a cell sample by contacting the cell lysate derived from the sample with a known quantity of fluorescently labeled O⁶—BG and using homogeneous fluorescence polarization techniques to measure the quantity of MGMT-bound O⁶—BG.

Materials and Methods

Exponentially growing cells are treated with trypsin and washed with PBS. Pelleted cells are resuspended with buffer (40 mM Tris-HCl, pH=8.0, 10% glycerol, 1 mM EDTA, 1 mM DTT, and protease inhibitors (Roche protease inhibitor cocktail tablets)) and lysed by sonication. Cell lysate is then centrifuged at 10,000K for 10 min.

Fluorescently labeled O⁶—BG (Covalys) at 10 mM is incubated with cell lysate for one hour. An instrument capable of reading fluorescence polarization is used to measure the fluorescence polarization value of the mixture. MGMT-bound O⁶—BG will have a higher polarization value than unreacted O⁶—BG. The fluorescence polarization value measured is used to calculate the amount of MGMT-bound O⁶—BG in the sample.

All patents, patent applications, accession numbers, publications, product descriptions, and protocols cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the invention. Accordingly, it is intended that the invention be limited only to the extent required by the appended claims and the applicable rules of law.

We claim:

1. A method for assessing the level of MGMT activity in a biological sample comprising:
(a) contacting the sample with O⁶—BG such that any MGMT in the sample can react with O⁶—BG to form a complex;
(b) detecting and quantitating the complex; and
(c) based on the quantity of the detected complex, determining the level of MGMT activity in the sample.

2. The method of claim 1, wherein detecting step (b) comprises immunoassay detection.

3. The method of claim 2, wherein the immunoassay detection comprises the steps of:
(1) coating the wells of a plate with the complex;
(2) treating the plate with an anti-MGMT antibody such that the anti-MGMT antibody binds to the complex; and
(3) detecting the bound anti-MGMT antibody in a fluorescent, enzymatic, chemiluminescent or radioactive assay system.

4. The method of claim 3, wherein the O⁶—BG is labeled with biotin and the plate is coated with avidin.

5. The method of claim 2, wherein the immunoassay detection comprises the steps of:
(1) coating the wells of a plate with the complex;
(2) treating the plate with an anti-MGMT antibody such that the anti-MGMT antibody binds to the complex;
(3) treating the plate with a secondary antibody such that the secondary antibody binds to the anti-MGMT antibody; and
(4) detecting the bound secondary antibody in a fluorescent, enzymatic, chemiluminescent or radioactive assay system.

6. The method of claim 5, wherein the O⁶—BG is labeled with biotin and the plate is coated with avidin.

7. A method for assessing the level of MGMT activity in a biological sample comprising:
(a) contacting the sample with O⁶—BG such that any MGMT in the sample can react with O⁶—BG to form a complex, wherein the benzyl group of O⁶—BG is labeled;
(b) separating unreacted O⁶—BG from the complex;
(c) detecting and quantitating the complex; and
(d) based on the quantity of the detected complex, determining the level of MGMT activity in the sample.

8. The method of claim 7, wherein step (b) comprises precipitation of the complex with acetone.

9. The method of claim 7, wherein step (b) comprises:
(1) adding the sample mixture to a plate coated with an anti-MGMT antibody, and
(2) washing the plate to remove unreacted O⁶—BG.

10. The method of claim 7, wherein the O⁶—BG label is fluorescent, enzymatic, chemiluminescent or radioactive.

11. The method of claim 10, wherein the O⁶—BG label is fluorescent and is selected from the group consisting of rare earth chelates, fluorescein, rhodamine, dansyl, dansyl chloride, umbelliferone, Lissamine, phycoerythrin, phycocyanin, allophycocyanin, o-phthaldehyde, fluorescamine, Texas Red, BODIPY, Alexa Fluors, Dyomics Dyes, Quasar Dyes, CY-dyes, and derivatives thereof.

12. The method of claim 10, wherein the O⁶—BG label is enzymatic and is selected from the group consisting of luciferases; 2,3-dihydrophthalazinediones; malate dehydrogenase; urease; peroxidases; alkaline phosphatase; beta-galactosidase; glucoamylase; lysozyme; saccharide oxidases; heterocyclic oxidases; acetylcholinesterase; lactoperoxidase and microperoxidase.

13. The method of claim 10, wherein the O⁶—BG label is chemiluminescent and is selected from the group consisting of luminol, isoluminol, theromatic acridinium ester, imidazole, acridinium salt, oxalate ester, luciferin and aequorin.

14. The method of claim 10, wherein the O⁶—BG label is radioactive and is selected from the group consisting of ³⁵S, ¹⁴C, ³H, ³²P, ¹²⁵I, ¹³¹I, ¹⁵N, ⁹⁰Y, ⁹⁹Tc and ¹¹¹In.

15. A method for assessing the level of MGMT activity in a biological sample comprising:
(a) contacting the sample with a known quantity of fluorescently labeled O⁶—BG such that any MGMT in the sample can react with O⁶—BG to form a complex;
(b) detecting fluorescence polarization indicative of complex formation, wherein the fluorescence polarization measurement indicates the quantity of the complex; and
(c) based on the quantity of the complex, determining the level of MGMT activity in the sample.

16. The method of claim 15, wherein the fluorescent label is selected from the group consisting of rare earth chelates, fluorescein, rhodamine, dansyl, dansyl chloride, umbelliferone, Lissamine, phycoerythrin, phycocyanin, allophycocyanin, o-phthaldehyde, fluorescamine, Texas Red, BODIPY, Alexa Fluors, Dyomics Dyes, Quasar Dyes, CY-dyes, and derivatives thereof.

17. The method of any of claims 1, 7, or 15, comprising the additional step of comparing the level of MGMT activity in the sample to that in
(a) a control sample exhibiting a high level of MGMT activity,
(b) a control sample exhibiting a low level of MGMT activity, or
(c) both (a) and (b).

18. The method of claim 17, wherein the control sample exhibiting a high level of MGMT activity is selected from the group consisting of an HT29 sample, a Capan-1 sample or a Capan-2 sample.

19. The method of claim 17, wherein the control sample exhibiting a low level of MGMT activity is an SNB19 sample.

20. The method of any of claims 1, 7, or 15, wherein the biological sample is a tissue or cell sample from lung, breast, ovary, stomach, pancreas, larynx, esophagus, testes, liver, parotid, biliary tract, colon, rectum, cervix, uterus, endometrium, kidney, spleen, bladder, prostate, thyroid, lymph node, pituitary, eye, brain, oral cavity, skin, bone, bone marrow, semen, stool, or a fraction or component thereof.

21. The method of any of claims 1, 7, or 15, wherein the biological sample is a tumor biopsy sample and the tumor type is prostate, breast, lung, pancreatic, colorectal, urinary system, NHL, melanoma, cervical, leukemia, oral cavity, ovarian, testicular, esophageal, liver, kidney, spleen, head and neck, carcinoma, sarcoma, lymphoma, mycosis fungoides or malignant glioma.

22. The method of claim 21, wherein the tumor biopsy sample is from bone marrow or lymph node and is from a patient suffering from leukemia.

23. The method of claim 21, wherein the tumor biopsy sample is from a high-grade tumor.

24. The method of any of claims 1, 7, or 15, wherein the alkylating agent is temozolomide, dacarbazine, busulfan, thiotepa, hydroxymethylmelamine, hexamethylmelamine, cyclophosphamide, mechlorethamine, uramustine, melphalan, chlorambucil, carmustine, streptozocin, lomustine, semustine, ifosfamide, porfiromycin, procarbazine, mitocycin C, cisplatin or carboplatin.

25. A kit comprising reagents and instructions for conducting the method according to any of claims 1, 7, or 15.

26. A method for predicting a chemotherapeutic efficacy of an alkylating agent in a patient in need thereof comprising assessing the level of MGMT activity in a biological sample from the patient by:
(a) contacting the sample with O⁶—BG such that any MGMT in the sample can react with O⁶—BG to form a complex;
(b) detecting and quantitating the complex; and
(c) based on the quantity of the detected complex, determining the level of MGMT activity in the sample;
wherein the predicted chemotherapeutic efficacy is inversely related to the level of MGMT activity in the sample.

27. The method of claim 26, wherein detecting step (b) comprises immunoassay detection.

28. The method of claim 27, wherein the immunoassay detection comprises the steps of:
(1) coating the wells of a plate with the complex;
(2) treating the plate with an anti-MGMT antibody such that the anti-MGMT antibody binds to the complex; and
(3) detecting the bound anti-MGMT antibody in a fluorescent, enzymatic, chemiluminescent or radioactive assay system.

29. The method of claim 28, wherein the O⁶—BG is labeled with biotin and the plate is coated with avidin.

30. The method of claim 27, wherein the immunoassay detection comprises the steps of:
(1) coating the wells of a plate with the complex;
(2) treating the plate with an anti-MGMT antibody such that the anti-MGMT antibody binds to the complex;
(3) treating the plate with a secondary antibody such that the secondary antibody binds to the anti-MGMT antibody; and
(4) detecting the bound secondary antibody in a fluorescent, enzymatic, chemiluminescent or radioactive assay system.

31. The method of claim 30, wherein the O⁶—BG is labeled with biotin and the plate is coated with avidin.

32. A method for predicting a chemotherapeutic efficacy of an alkylating agent in a patient in need thereof comprising assessing the level of MGMT activity in a biological sample from the patient by:
(a) contacting the sample with O⁶—BG such that any MGMT in the sample can react with O⁶—BG to form a complex, wherein the benzyl group of O⁶—BG is labeled;
(b) separating unreacted O⁶—BG from the complex;
(c) detecting and quantitating the complex; and
(d) based on the quantity of the detected complex, determining the level of MGMT activity in the sample;
wherein the predicted chemotherapeutic efficacy is inversely related to the level of MGMT activity in the sample.

33. The method of claim 32, wherein step (b) comprises precipitation of the complex with acetone.

34. The method of claim 32, wherein step (b) comprises:
(1) adding the sample mixture to a plate coated with an anti-MGMT antibody, and
(2) washing the plate to remove unreacted O⁶—BG.

35. The method of claim 32, wherein the O⁶—BG label is fluorescent, enzymatic, chemiluminescent or radioactive.

36. The method of claim 35, wherein the O⁶—BG label is fluorescent and is selected from the group consisting of rare earth chelates, fluorescein, rhodamine, dansyl, dansyl chloride, umbelliferone, Lissamine, phycoerythrin, phycocyanin, allophycocyanin, o-phthaldehyde, fluorescamine, Texas Red, BODIPY, Alexa Fluors, Dyomics Dyes, Quasar Dyes, CY-dyes, and derivatives thereof.

37. The method of claim 35, wherein the O⁶—BG label is enzymatic and is selected from the group consisting of luciferases; 2,3-dihydrophthalazinediones; malate dehydrogenase; urease; peroxidases; alkaline phosphatase; beta-galactosidase; glucoamylase; lysozyme; saccharide oxidases; heterocyclic oxidases; acetylcholinesterase; lactoperoxidase and microperoxidase.

38. The method of claim 35, wherein the O⁶—BG label is chemiluminescent and is selected from the group consisting of luminol, isoluminol, theromatic acridinium ester, imidazole, acridinium salt, oxalate ester, luciferin and aequorin.

39. The method of claim 35, wherein the O⁶—BG label is radioactive and is selected from the group consisting of ³⁵S, ¹⁴C, ³H, ³²P, ¹²⁵I, ¹³¹I, ¹⁵N, ⁹⁰Y, ⁹⁹Tc and ¹¹¹In.

40. A method for predicting a chemotherapeutic efficacy of an alkylating agent in a patient in need thereof comprising assessing the level of MGMT activity in a biological sample from the patient by:
(a) contacting the sample with a known quantity of fluorescently labeled O⁶—BG such that any MGMT in the sample can react with O⁶—BG to form a complex;
(b) detecting fluorescence polarization indicative of complex formation, wherein the fluorescence polarization measurement indicates the quantity of the complex; and
(c) based on the quantity of the complex, determining the level of MGMT activity in the sample;
wherein the predicted chemotherapeutic efficacy is inversely related to the level of MGMT activity in the sample.

41. The method of claim 40, wherein the fluorescent label is selected from the group consisting of rare earth chelates, fluorescein, rhodamine, dansyl, dansyl chloride, umbelliferone, Lissamine, phycoerythrin, phycocyanin, allophycocyanin, o-phthaldehyde, fluorescamine, Texas Red, BODIPY, Alexa Fluors, Dyomics Dyes, Quasar Dyes, CY-dyes, and derivatives thereof.

42. The method of any of claims 26, 32, or 40, comprising the additional step of comparing the level of MGMT activity in the sample to that in
(a) a control sample exhibiting a high level of MGMT activity,
(b) a control sample exhibiting a low level of MGMT activity, or
(c) both (a) and (b).

43. The method of claim 42, wherein the control sample exhibiting a high level of MGMT activity is selected from the group consisting of an HT29 sample, a Capan-1 sample or a Capan-2 sample.

44. The method of claim 42, wherein the control sample exhibiting a low level of MGMT activity is an SNB19 sample.

45. The method of any of claims 26, 32, or 40, wherein the biological sample is a tissue or cell sample from lung, breast, ovary, stomach, pancreas, larynx, esophagus, testes, liver, parotid, biliary tract, colon, rectum, cervix, uterus, endometrium, kidney, spleen, bladder, prostate, thyroid, lymph node, pituitary, eye, brain, oral cavity, skin, bone, bone marrow, semen, stool, or a fraction or component thereof.

46. The method of any of claims 26, 32, or 40, wherein the biological sample is a tumor biopsy sample and the tumor type is prostate, breast, lung, pancreatic, colorectal, urinary system, NHL, melanoma, cervical, leukemia, oral cavity, ovarian, testicular, esophageal, liver, kidney, spleen, head and neck, carcinoma, sarcoma, lymphoma, mycosis fungoides or malignant glioma.

47. The method of claim 46, wherein the tumor biopsy sample is from bone marrow or lymph node and is from a patient suffering from leukemia.

48. The method of claim 46, wherein the tumor biopsy sample is from a high-grade tumor.

49. The method of any of claims 26, 32, or 40, wherein the alkylating agent is temozolomide, dacarbazine, busulfan, thiotepa, hydroxymethylmelamine, hexamethylmelamine, cyclophosphamide, mechlorethamine, uramustine, melphalan, chlorambucil, carmustine, streptozocin, lomustine, semustine, ifosfamide, porfiromycin, procarbazine, mitocycin C, cisplatin or carboplatin.

50. A kit comprising reagents and instructions for conducting the method according to any of claims 26, 32, or 40.
